(19) United States
(12) Patent Application Publication
(10) Pub. No.: US 2023/0329311 A1
(43) Pub. Date: Oct. 19, 2023

(54) METHOD FOR SHAPING A CELL-MASS MIXTURE BY VACUUM SEALING

(71) Applicant: Upside Foods, Inc., Berkeley, CA (US)

(72) Inventor: Antony John Wicke, San Leandro, CA (US)

(21) Appl. No.: 17/659,807

(22) Filed: Apr. 19, 2022

(51) Int. Cl. A23P 30/10; C12N 5/077; A23L 13/00; A23L 29/00

(52) U.S. Cl. CPC A23P 30/10; C12N 5/0658; A23L 13/00; A23L 29/06; A23V 2002/00

(57) ABSTRACT

This disclosure relates to methods of shaping a raw cell-based-meat product using vacuum sealing. The disclosed method includes preparing a cell-mass mixture by mixing a grown cell mass with a binding agent. The cell-mass mixture is added to a mold that is covered with a film and vacuum sealed. Because, in some embodiments, the mold is rigid, the cell-mass mixture conforms to the shape of the mold, such as the shape of slaughtered or harvested meat. To shape a cell-based-meat product, an apparatus may be used with a mold for sealing and conforming a cell-mass mixture.

### BACKGROUND

As the world's population continues to grow, cell-based or cultured meat products for consumption have emerged as an attractive alternative (or supplement) to conventional meat from animals. For instance, cell-based, cultivated, or cultured meat represents a technology that could address the specific dietary needs of humans. Cell-based meat products can be prepared from a combination of cultivated adherent and suspension cells derived from a non-human animal. Because the cells for cell-based meat are lab grown, cell masses are often formed and shaped to mimic familiar forms of conventional meat.

In addition to addressing dietary needs, cell-based-meat products help alleviate several drawbacks linked to conventional meat products for humans, livestock, and the environment. For instance, conventional meat production involves controversial practices associated with animal husbandry and slaughter. Other drawbacks associated with conventional meat production include low conversion of caloric input to edible nutrients, microbial contamination of the product, emergence and propagation of veterinary and zoonotic diseases, relative natural resource requirements, and resultant industrial pollutants, such as greenhouse gas emissions and nitrogen waste streams.

Despite advances in creating cell-based-meat products, existing methods for cultivating and processing cell-based-meat products face several shortcomings, such as challenges or failures to shape cell-based-meat products. More particularly, existing methods of processing cell-based-meat products often result in shapeless cell-based-meat masses. To illustrate, cell-based-meat products, especially cells grown in suspension, often exhibit no distinct form but rather a pliable and shape-shifting mass. At most, existing methods create roughly shaped cell-based-meat products.

Furthermore, existing methods for cultivating and processing cell-based-meat products often fail to mimic the textures of conventional meat—both internally and externally. As one example, existing methods often result in cell-based-meat products that contain sporadic air pockets within the products. Externally, existing methods often fail to create products with surface textures like conventional meat. Accordingly, existing methods are often limited to creating cell-based-meat products that mimic highly processed conventional meats that suffer from the same textural deficiencies.

These, along with additional problems and issues are present in existing methods for cultivating cell-based-meat products.

### BRIEF SUMMARY

This disclosure generally describes methods and apparatuses of shaping a cell-based-meat product to conform to a mold by utilizing vacuum forming. In particular, the disclosed method includes combining a cell tissue with a binder. The disclosed method further includes portioning the tissue-binder mixture and placing the mixture into a mold. The mold may exhibit a shape of slaughtered or harvested meat, such as a chicken-breast shape, a chicken-nugget shape, patties, thigh meat, a top loin steak, or a lobster claw. The mold and mixture are covered with a film and vacuum sealed. When taken out of the mold, the mixture retains the shape of the mold. By conforming to a shape of a mold, the cell-based-meat product can resemble slaughtered or harvested meat and remove air pockets.

To shape a cell-based-meat product, an apparatus may be used with a mold. Generally, the apparatus includes a portioner that optionally portions a cell-mass mixture and deposits the cell-mass mixture into the mold. In some embodiments, the apparatus also includes a film applicator that applies a film to the mold and a vacuum sealer that seals the mold to shape the cell-mass mixture to the mold.

### BRIEF DESCRIPTION OF THE DRAWINGS

The detailed description refers to the drawings briefly described below.

FIG. 1 illustrates an overview diagram of shaping a cell-mass mixture using vacuum sealing in accordance with one or more embodiments of the present disclosure.

FIG. 2 illustrates example characteristics of a shapeless cell-mass mixture, a mold, and a shaped cell-mass mixture in accordance with one or more embodiments of the present disclosure.

FIG. 3 illustrates an overview diagram of preparing a grown cell mass in accordance with one or more embodiments of the present disclosure.

FIG. 4 illustrates an example apparatus comprising various components for portioning a grown cell mass into a mold, covering the mold, and vacuum sealing the mold in accordance with one or more embodiments of the present disclosure.

FIGS. 5A-5B illustrate example molds in accordance with one or more embodiments of the present disclosure.

FIGS. 6A-6B illustrate a front and back view of an example film envelope enclosing a cell-mass mixture within a mold in accordance with one or more embodiments.

FIGS. 7-8 illustrate series of acts for shaping a cell-mass mixture using vacuum sealing in accordance with one or more embodiments of the present disclosure.

### DETAILED DESCRIPTION

This disclosure describes one or more embodiments of a method for vacuum forming a grown cell mass to conform to the shape and contours of a mold. The disclosed method includes mixing tissue with a binder and placing the mixture in a mold having a desired shape and surface texture. The disclosed method further includes covering the mold with a film and vacuum sealing the mixture and the mold. The resulting cell-based-meat product has a high fidelity of shape and texture to the mold.

To illustrate, the disclosed method comprises mixing a grown cell mass and a binding agent to form a cell-mass mixture. The disclosed method further comprises adding the cell-mass mixture to a mold and covering the cell-mass mixture and the mold with a film. The disclosed method includes vacuum sealing the cell-mass mixture within the mold to shape the cell-mass mixture.

As mentioned, the disclosed method includes mixing a grown cell mass and a binding agent to form a cell-mass mixture. Generally, the binding agent associates with cells in the cell-mass mixture to retain its shape. In some embodiments, the binding agent and the grown cell mass are simultaneously cut and mixed.

The disclosed method further comprises placing the shapeless cell-mass mixture into a mold. The mold can have any shape or surface texture. For example, a mold can have the shape and surface texture of a chicken breast, beef steak, lobster claw, or another form. In some embodiments, the mold is made of a rigid material. In some embodiments, the disclosed method includes portioning the cell-mass mixture before placing the cell-mass mixture in the mold. For instance, the method can include portioning the cell-mass mixture into portions of a particular weight (e.g., 4 oz., 7 oz.) or portioning the cell-mass mixture into thirds, fourths, or some other fraction and subsequently pacing the portioned cell-mass mixtures into respective molds.

After placing a cell-mass mixture into a mold, in some embodiments, the disclosed method comprises covering the cell-mass mixture and the mold with a film. The film seals the cell-mass mixture within the mold. In some examples, the film comprises a thick material that retains its shape.

After covering the mold and cell-mass mixture with film, the disclosed method comprises vacuum sealing the cell-mass mixture within the mold. More particularly, the disclosed method includes evacuating air from within the mold. Accordingly, the disclosed method reduces or removes air bubbles from the cell-mass mixture. The vacuum sealing also shapes the cell-mass mixture to conform to the mold.

The following disclosure also describes an apparatus that shapes the cell-mass mixture to the mold. In particular, the disclosed apparatus includes a portioner. In some embodiments, the portioner weighs and divides a cell-mass mixture. The portioner further deposits the cell-mass mixture into a rigid mold.

The disclosed apparatus may further include a film applicator and a vacuum sealer. The film applicator covers the rigid mold and the cell-mass mixture with a film. The film applicator covers the rigid mold as part of creating a seal. The vacuum sealer removes air from the covered rigid mold so the cell-mass mixture conforms to the shape of the mold.

The disclosed method provides several benefits relative to unprocessed cell cultures or other existing and unprocessed cell-based meats. By vacuum sealing the cell-mass mixture in a mold, the disclosed method shapes the cell-mass mixture. Likewise, the apparatus can include a rigid mold, film applicator, and vacuum sealer that shape and seal a cell-mass mixture to conform to the mold and give the cell-mass mixture a distinctive shape, such as a shape mimicking slaughtered or harvested meat. The cell-mass mixture conforms to the shape of the mold. Additionally, by vacuum sealing the cell-mass mixture in the mold, the disclosed method ensures that the outer portion of the cell-mass mixture conforms to the mold, even if it has a surface texture. Accordingly, the disclosed method can shape a grown cell mass with greater precision than existing methods.

In addition to shaping a cell-based-meat product, the disclosed method also improves the texture of the cell-based-meat product relative to existing cell-based meats. In particular, vacuum sealing not only shapes the cell-mass mixture but also removes air bubbles from within the cell-mass mixture. Thus, the disclosed method can control and improve the texture of cell-based meat to better mimic cuts of slaughtered meat. Similarly, the disclosed apparatus can remove air bubbles and likewise improve the texture of cell-based meat. Furthermore, in some embodiments, the disclosed method improves the texture of the cell-based-meat product by adding surface texture to the final product.

As illustrated by the foregoing discussion, the present disclosure utilizes a variety of terms to describe features and advantages of the disclosed method. Additional detail is now provided regarding the meaning of such terms. For example, as used herein, the term “cell mass” refers to a mass comprising cells of meat. In particular, a cell mass refers to cells of cultured meat gathered into a collective mass. In some embodiments, the cell mass is comestible. As discussed below, a cell mass may comprise different cell types, such as one or more of myoblasts, mesangioblasts, myofibroblasts, mesenchymal stem cells, hepatocytes, fibroblasts, pericytes, adipocytes, epithelial, chondrocytes, osteoblasts, osteoclasts, pluripotent cells, somatic stem cells, endothelial cells, or other similar cell types. For example, a cell mass can include a cell sheet of cultured meat growing within an enclosure, such as a chamber, housing, container, etc.

Relatedly, the term “grown cell mass” refers to a cell mass comprising one or more grown cells. For instance, a grown cell mass includes a group of cells that have been nourished by a growth medium (e.g., a cell culture medium) to grow during a growing time period. In some cases, a grown cell mass includes a cell mass that has finished a growing time period.

As further used herein, the term “cells” refers to individual cells of meat. In particular, cells may comprise different cell types, such as one or more of myoblasts, mesangioblasts, myofibroblasts, mesenchymal stem cells, hepatocytes, fibroblasts, pericytes, adipocytes, epithelial, chondrocytes, osteoblasts, osteoclasts, pluripotent cells, somatic stem cells, endothelial cells, or other similar cell types. Furthermore, cells may comprise different types of progenitor cells, including myogenic progenitors, adipogenic progenitors, mesenchymal progenitors, or other types of progenitor cells.

As used herein, the term “binding agent” refers to a material or substance that holds or draws other materials together. In particular, a binding agent refers to a substance that draws cells together to form a cohesive whole mechanically, chemically, by adhesion, or cohesion. Binding agents can include enzymes, gums, extracts, proteins, and other substances. More specifically, binding agents may include transglutaminase, modified starches, carrageenan, soluble and insoluble fibers, gelatin, sodium caseinate, wheat gluten, dry milk, protein, and others. Binding agents may include gums, such as guar or xanthan.

As further used herein, the term “cell-mass mixture” refers to a mixture comprising cells and other substances. In particular, a cell-mass mixture comprises a combination of a grown cell mass and one or more binding agents. For example, a cell-mass mixture may comprise a grown cell mass and transglutaminase that have been mixed using a bowl chopper.

As used herein, the term “mold” refers to a hollow container used to give shape to a material. In particular, a mold includes a container having a shape of a desired meat product. For example, a mold may comprise a food-safe container in the shape of a chicken breast, chicken nugget, a top loin steak, a lobster claw, or a shank crosscut. The mold may additionally have surface texture that mimics the desired meat product.

As used herein, the term “film” refers to a thin continuous material. In particular, a film comprises a material that, when applied to a mold, forms an airtight seal. For example, a film may comprise laminated films, vacuum bags, thermoforming films made of various materials. A film may comprise a flexible polymer.

As used herein, the term “vacuum sealing” refers to a method for sealing a container by evacuating air from the container. In particular, vacuum sealing refers to the process of removing air from a cell-mass mixture and a mold.

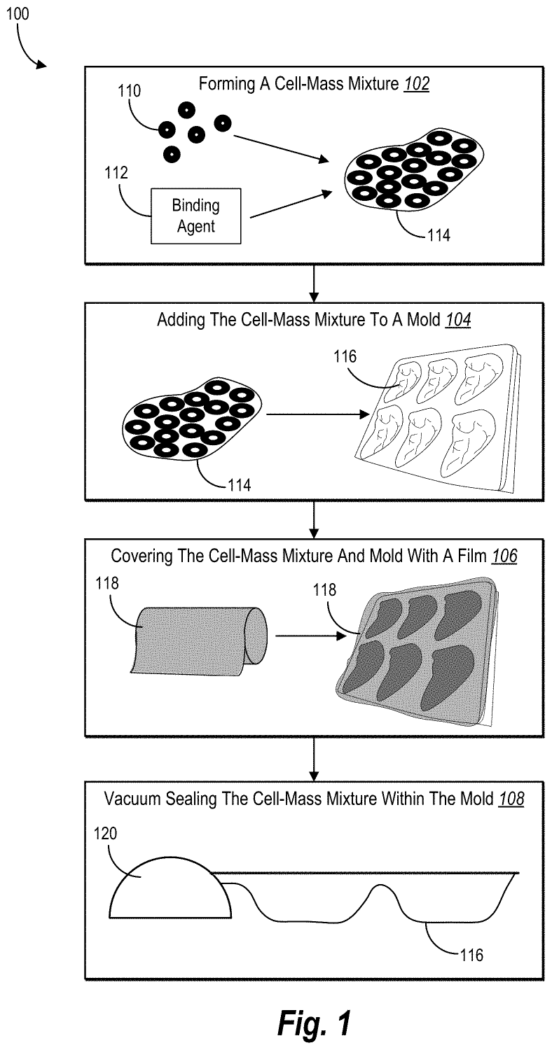

Additional detail will now be provided regarding a disclosed method in relation to illustrative figures portraying example embodiments and implementations of the disclosed methods and apparatuses. For example, FIG. 1 illustrates an overview of acts in the disclosed method to shape a cell-mass mixture using vacuum sealing in accordance with one or more embodiments. FIG. 1 illustrates a series of acts 100 including an act 102 of forming a cell-mass mixture, an act 104 of adding the cell-mass mixture to a mold, an act 106 of covering the cell-mass mixture and mold with a film, and an act 108 of vacuum sealing the cell-mass mixture within the mold.

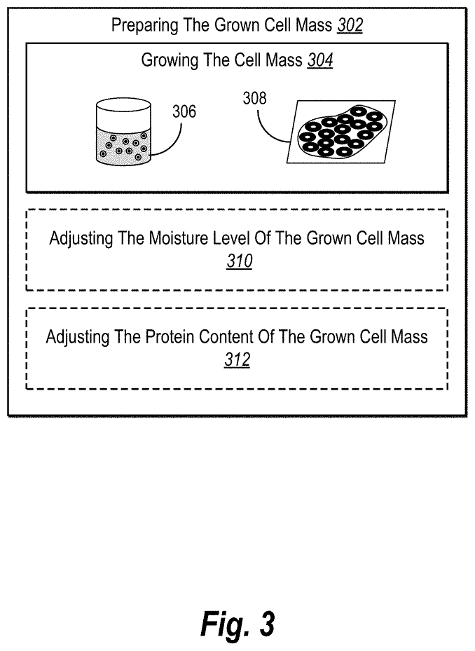

As illustrated in FIG. 1, the disclosed method includes the act 102 of forming a cell-mass mixture. In particular, the act 102 comprises mixing a grown cell mass 110 and a binding agent 112 to form a cell-mass mixture 114. With time, the binding agent 112 draws the grown cell mass 110 together so the cell-mass mixture 114 will hold its shape after being formed within a mold. To illustrate, in some embodiments, the binding agent 112 constitutes transglutaminase. The binding agent 112 may include one or more substances, as described above. In some implementations, the disclosed method includes forming the cell-mass mixture 114 by blending the grown cell mass 110 with the binding agent 112. In some embodiments, the disclosed method comprises preparing the grown cell mass 110 before combining the grown cell mass 110 with the binding agent 112. FIG. 3 and the corresponding paragraphs describe preparing the grown cell mass in accordance with one or more embodiments of the disclosed method.

In some embodiments, the binding agent 112 provides mechanical structure for the cell-mass mixture. For example, the binding agent 112 may comprise some sort of scaffolding. In some embodiments, the binding agent 112 comprises gelatin or collagen.

In some embodiments, the disclosed method includes combining the grown cell mass 110 with additional supplements. In particular, supplements may be added to the grown cell mass 110 to improve texture. For example, the disclosed method may include the addition of a drying agent, such as dried protein powder, which helps bind the cell-mass mixture 114 together more firmly. In some embodiments, different supplements are added based on whether the grown cell mass 110 comprises adherent or suspension cells. In particular, cells grown in suspension tend to contain more moisture than adherent cells. Adherent cells may be more easily shaped than wet suspension cells. In one example, suspension cells are combined with additional plant proteins, stabilizers (e.g., methylcellulose), gums, modified starches, and/or native starches in a vacuum blender to improve the firmness and overall texture of the cell-mass mixture 114.

In some implementations, the grown cell mass 110 comprises a raw or uncooked grown cell mass. More specifically, the process of vacuum sealing is meant to shape a raw or uncooked cell-based-meat product. The uncooked shaped cell-based-meat product may further be cooked in the mold or cooked after removal from the mold. Accordingly, in some cases, the cell-based-meat product is comestible.

In one example, the disclosed method performs the act 102 utilizing a bowl chopper. In particular, the grown cell mass 110 is run through a bowl chopper that reduces the size of tissue in the grown cell mass 110. The bowl chopper acts as a dumb cutter and runs blades through the grown cell mass 110 until the grown cell mass 110 is ground or comminuted. In some cases, the disclosed method further comprises collecting the grown cell mass 110 and combining the grown cell mass 110 with the binding agent 112 in a vacuum blender, which reduces air bubbles and homogenizes the cell-mass mixture 114. By contrast, in some implementations, the disclosed method does not cut (e.g., using a bowl chopper) cells grown in suspension.

After forming a cell-mass mixture, FIG. 1 further illustrates the act 104 of adding the cell-mass mixture to a mold. As illustrated, the cell-mass mixture 114 is added to a mold 116. As shown in FIG. 1, the mold 116 is part of a sheet of molds for respective cell-mass mixtures. In some implementations, the disclosed method includes portioning the cell-mass mixture 114 based on weight and placing the portions into the mold 116. For example, the cell-mass mixture 114 may be separated into serving portions of predetermined weights (e.g., 4 oz.). The shapeless portions are placed into the mold 116. As illustrated, in some embodiments, the mold 116 has a flat surface to which a film adheres.

The mold 116 illustrated in FIG. 1 has both a desired shape and surface texture. As illustrated, the mold 116 has the shape and surface texture of a chicken breast. The disclosed method may utilize molds having different shapes to mimic conventional meat shapes. For example, molds may have shapes of chicken tenders, nuggets, patties, thigh meat, wing meat, boneless drumsticks, etc. Molds may also mimic other types of meat beside chicken. For instance, the disclosed system may utilize molds with the shape of fish filets, lobster claws, beef steaks, and other forms of slaughtered or harvested meat.

In some embodiments, the disclosed method creates the mold 116 by utilizing a master mold. Generally, the disclosed method includes creating the mold 116 by shaping a sheet to conform to the shape and contours of the master mold. The sheet may be made of heat and food safe material so the cell-mass mixture 114 may be heated and cooked within the mold 116 after shaping. In one example, the disclosed method forms the mold 116 by heating the sheet, stretching the sheet over the mold 116, and blowing the sheet into the mold 116. For instance, the disclosed method may comprise heating the sheet to a temperature between 176 F-311 F. When the sheet cools, it hardens into the mold 116 that conforms to the shape and surface texture of the master mold. In some embodiments, the mold 116 is a rigid mold to which the cell-mass mixture 114 conforms. While some conventional vacuum sealing methods vacuum seal pliable wrappers around products during packaging, the disclosed method utilizes a rigid mold to which the cell-mass mixture 114 conforms during vacuum sealing.

After adding the cell-mass mixture to a mold, FIG. 1 further illustrates the act 106 of covering the cell-mass mixture and mold with a film. Generally, the disclosed system covers the mold 116 with a film 118. In some embodiments, the material of the film 118 is thinner and less rigid than the material used to make the mold 116. For example, the film 118 may be 12 millimeters in thickness. The film 118 is rolled onto the mold 116. For instance, the film 118 may be rolled onto a flat surface of the mold 116.

As further illustrated in FIG. 1, the disclosed method includes an act 108 of vacuum sealing the cell-mass mixture within the mold. Generally, the disclosed method utilizes a vacuum sealer 120 to seal and shape the cell-mass mixture to the mold 116. During vacuum sealing, the vacuum sealer 120 removes the air within the mold 116 and leaves the cell-mass mixture 114 intact. By vacuum sealing the cell-mass mixture 114, the disclosed method removes air pockets within the cell-mass mixture 114. Additionally, vacuum sealing also removes air between an outer portion of the cell-mass mixture 114 and the mold 116. Accordingly, in some cases, the outer portion of the cell-mass mixture 114 conforms with greater fidelity to the surface texture of the mold 116.

In some embodiments, the series of acts 100 further comprises placing the vacuum sealed cell-mass mixture in a temperature-controlled environment for a threshold time. Generally, binding agents form connections between cells in the grown cell mass 110 over time. Accordingly, in some embodiments, the disclosed method includes vacuum sealing the cell-mass mixture 114 and the mold 116 before the binding agent 112 has completed making connections such that the vacuum forces allow a shapeless cell-mass mixture to conform (or take on) the shape of the mold. Either in a temperature-controlled environment or elsewhere, in some embodiments, the cell-mass mixture 114 is left to rest so that the binding agent 112 has sufficient time to bind cells together and accordingly allow the cell-based meat product to retain the shape of the mold once the cell-based meat product is removed.

In particular, in some embodiments, the threshold time equals the time required for the cell-mass mixture 114 to set under the vacuum pressure. The threshold time may be 12 hours, 24 hours, 36 hours, etc. During this time, the cell-mass mixture 114 takes on the shape and the surface texture of the mold 116. Furthermore, because the cell-mass mixture 114 is a meat product, the sealed cell-mass mixture is stored within a temperature-controlled environment (e.g., a refrigerator). In one example, the disclosed method includes storing the sealed cell-mass mixture for twenty-four hours at a refrigerated temperature between 34 F-40 F.

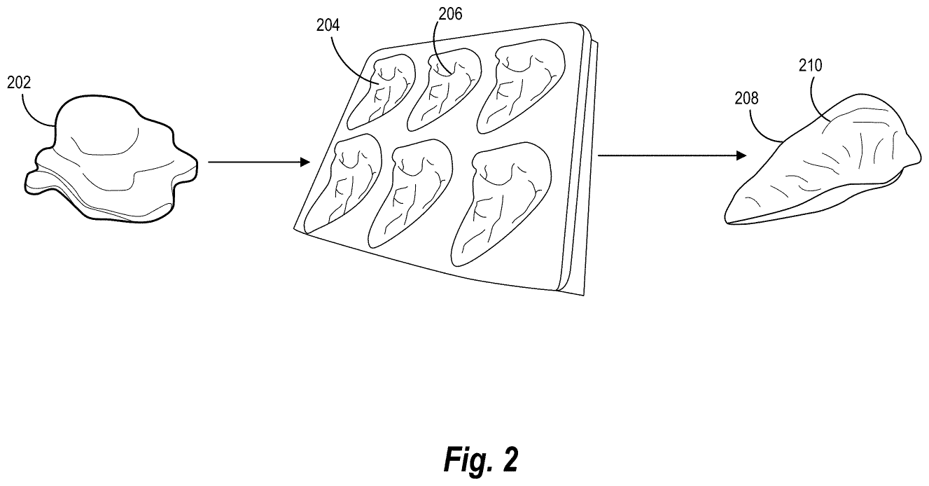

As noted above, the disclosed method can shape a pliable cell-mass mixture by placing the cell-mass mixture into a mold to which the mixture conforms. FIG. 2 illustrates how the disclosed method utilizes vacuum shaping to form a shapeless cell-mass mixture into a cell-based meat product in accordance with one or more embodiments. In particular, FIG. 2 illustrates a shapeless cell-mass mixture 202. As mentioned, the shapeless cell-mass mixture 202 comprises a combination of a grown cell mass and a binding agent. The shapeless cell-mass mixture 202 is pliable and not yet formed.

As further shown in FIG. 2, in some embodiments, the disclosed method includes portioning the shapeless cell-mass mixture 202 and depositing the shapeless cell-mass mixture 202 into a mold 204. In this example, the mold 204 is part of a sheet or tray of molds for respective cell-mass mixtures. As depicted in FIG. 2, the mold 204 has surface texture 206. The surface texture 206 may be designed to impart features of conventional meat on a cell-based-meat product 208. For example, the surface texture 206 can resemble striations on a cut of slaughtered meat. The disclosed method vacuum seals the mold 204 and the shapeless cell-mass mixture 202, and the shapeless cell-mass mixture 202 conforms to the shape of the mold 204.

FIG. 2 illustrates the cell-based-meat product 208 conforming to a surface texture 210 to exhibit the surface texture 210. More specifically, the surface texture 210 comes from the surface texture 206 of the mold 204. In addition to conforming to the surface texture 210, the cell-based-meat product 208 also conforms to a general shape of the mold 204.

In some embodiments, the mold 204 and the film make up the packaging for the cell-based-meat product 208. For example, and as previously mentioned, the mold 204 and the film can be heat rated so that the cell-based-meat product 208 may be cooked within the mold 204. In one example, the mold 204, cell-based-meat product 208, and film are immersed in hot water to cook the cell-based-meat product 208. In other embodiments, the cell-based-meat product 208 is removed from the mold 204 and further processed. In one example, the cell-based-meat product 208 is removed from the mold 204 and cooked, battered, fried, etc.

Typically, vacuum sealing in packaging is used to preserve a pre-shaped item. Some existing methods pre-form food products by cutting the food products into 2D or 3D shapes. Fully cooked and breaded meat items (e.g., nuggets, patties) and raw portioned meat parts (e.g., chicken breast, steak), are placed in a deep well package having a circular or rectangular shape. The deep well package is vacuum sealed and covered with plastic. Because the aforementioned meat products are fully formed and relatively rigid, their 3D shape is not affected by vacuum packaging, and the aforementioned meat products do not conform to the shape of their mold. In contrast, the disclosed method uses vacuum sealing to form a raw cell-based-meat product to the shape of a rigid mold.

As mentioned previously, the disclosed method comprises preparing a grown cell mass. FIG. 3 and the corresponding paragraphs describe how a grown cell mass is prepared in accordance with one or more embodiments. In particular, FIG. 3 illustrates an act 302 of preparing the grown cell mass. As part of the act 302, the disclosed method includes an act 304 of growing the cell mass, an optional act 310 of adjusting the moisture level of the grown cell mass, and an optional act 312 of adjusting the protein content of the grown cell mass.

As illustrated in FIG. 3, the disclosed method comprises the act 304 of growing the cell mass. The grown cell mass may comprise suspension cells 306 in which cells are grown in an agitated growth medium, thus forming a suspension. The grown cell mass may also comprise adherent cells 308, which are cells grown while attached to a surface. In either case, the disclosed method may comprise growing cells within a growth environment, such as a bioreactor. Both the suspension cells 306 and the adherent cells 308 may be grown in the presence of cell culture media.

After growing the cell mass, FIG. 3 further illustrates the optional act 310 of adjusting the moisture level of the grown cell mass. Generally, the grown cell mass needs a low moisture content to be processed and shaped. Wet tissue is more difficult to form. Because suspension cells 306 have higher moisture content, the disclosed method includes drying the suspension cells 306. The suspension cells 306 may be dehydrated by mechanical means, for example, by straining the grown cell mass from fluid. In some embodiments, the optional act 310 includes adding a drying agent, such as dried protein powder to the suspension cells 306. Dried protein powder provides an added benefit of more firmly binding the product. In some embodiments, the disclosed method performs the optional act 310 for the suspension cells 306 but omits the optional act 310 for the adherent cells 308. In other embodiments, the disclosed method performs the optional act 310 for both the adherent cells 308 and the suspension cells 306.

In some examples, due in part to the difficulty of forming the suspension cells 306, even after dehydration, cell-based-meat products stemming from the suspension cells 306 and the adherent cells 308 may be used differently. For example, the adherent cells 308 may support more premium products because the texture is closer to that of slaughtered or harvested meat. Thus, cell-based-meat products made from the suspension cells 306 may be further processed, for example, by breading and frying.

As further illustrated in FIG. 3, the disclosed method may include the optional act 312 of adjusting the protein content of the grown cell mass. Generally, some binding agents, such as transglutaminase, require the cell-mass mixture to have a threshold protein content. For example, a grown cell mass having less than 15% protein content may not react with transglutaminase to successfully hold its shape. Accordingly, in some embodiments, the disclosed method includes increasing the protein content of the grown cell mass prior to processing. The disclosed method may do so, for example, by combining the grown cell mass with protein additives or by further concentrating the grown cell mass by dehydrating the grown cell mass. The disclosed method can also adjust the protein content to meet threshold protein contents of other binding agents. For instance, gums may require a lower protein content (e.g., 5%) than the protein content required by transglutaminase.

In some implementations, the disclosed method excludes the optional act 312. While some binding agents require a threshold protein content level, other binders do not. Thus, in some embodiments, the disclosed method does not adjust the protein content of the grown cell mass.

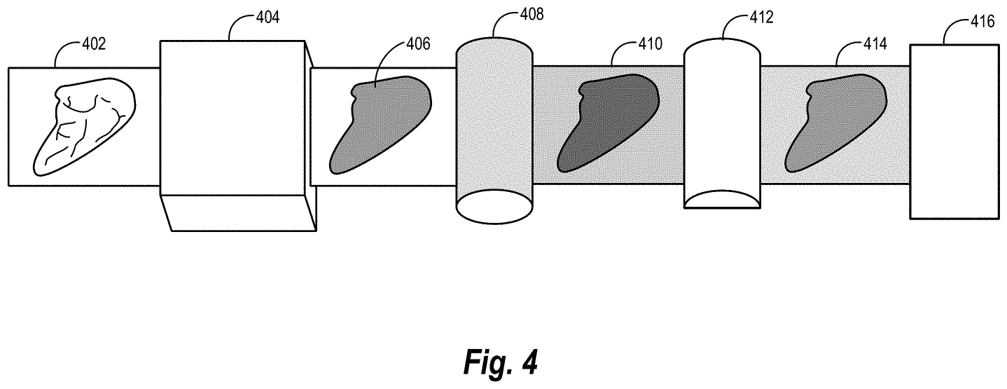

To shape a cell-based-meat product, an apparatus may be used with a mold. FIG. 4 illustrates an apparatus for shaping and vacuum sealing a cell-mass mixture in accordance with one or more embodiments. In particular, FIG. 4 illustrates an apparatus comprising a portioner 404, a rigid mold 402, a film applicator 408, a vacuum sealer 412, and a temperature-controlled environment 416. By way of overview, a cell-mass mixture is deposited in the rigid mold 402, the film applicator 408 applies a film, and the vacuum sealer 412 seals the film to the mold.

As illustrated in FIG. 4, the apparatus includes the portioner 404. The portioner 404 generally deposits a cell-mass mixture into the rigid mold 402. In particular, the portioner 404 collects a cell-mass mixture, weighs the mixture, and portions out the mixture before depositing the portions into molds. In some embodiments, the portioner 404 comprises a vacuum pump to weigh and portion out the mixture. The portioner 404 weighs and divides the cell-mass mixture to deposit a portion 406 of the cell-mass mixture in the rigid mold 402.

As further illustrated in FIG. 4, the apparatus also includes the film applicator 408. The film applicator 408 covers the rigid mold 402 and the portion 406 with a film. In some embodiments, the film applicator 408 seals or partially seals the rigid mold 402. For example, the film applicator 408 may heat a film and stretch the film over the rigid mold 402. In another example, the film applicator 408 heats and presses segments of the film onto the rigid mold 402. As the film cools, the film adheres to the rigid mold 402. In other embodiments, the film applicator 408 adheres film to the rigid mold 402 using an adhesive. In some implementations, the film applicator 408 simply covers the mold using the film and the film is sealed to the rigid mold 402 after vacuum sealing. For example, the covered mold 410 represents the rigid mold 402 holding a portion 406 of cell-mass mixture covered by a film.

FIG. 4 further illustrates the vacuum sealer 412. The vacuum sealer 412 removes air from the covered rigid mold holding the cell-mass mixture. In particular, the vacuum sealer 412 seals and shapes the cell-mass mixture to form a vacuum sealed cell-mass mixture 414. In some embodiments, the vacuum sealer 412 seals the packaging comprising the rigid mold 402, the portion 406, and the film. In some embodiments, the disclosed method utilizes a joint film applicator and vacuum sealer that simultaneously vacuum seals the rigid mold 402 while applying the film.

In some embodiments, the apparatus includes an inkjet printer for printing on the film. For example, in embodiments, where the film and the mold comprise final packaging for the cell-based-meat product, an inkjet printer may print the packaging with information. Additionally, the apparatus may include a cutter for cutting the film and/or separating individual molds from a sheet of molds.

As further illustrated in FIG. 4, in some embodiments, the apparatus includes the temperature-controlled environment 416. While the temperature-controlled environment 416 may be part of and directly connected to the apparatus, in some embodiments, the temperature-controlled environment 416 is separate from and not physically connected to the apparatus. Generally, the temperature-controlled environment 416 controls the temperature within an environment, such as a refrigerator. The vacuum sealed cell-mass mixture 414 is stored in the temperature-controlled environment 416 for a threshold time to allow the cell-mass mixture to set in the rigid mold 402. As mentioned, the vacuum sealed cell-mass mixture 414 may be stored in the temperature-controlled environment for 12 hours, 24 hours, etc.

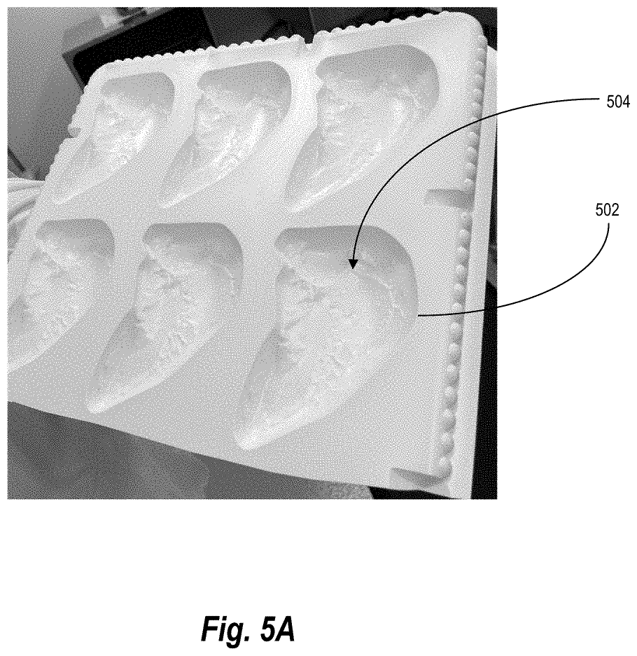

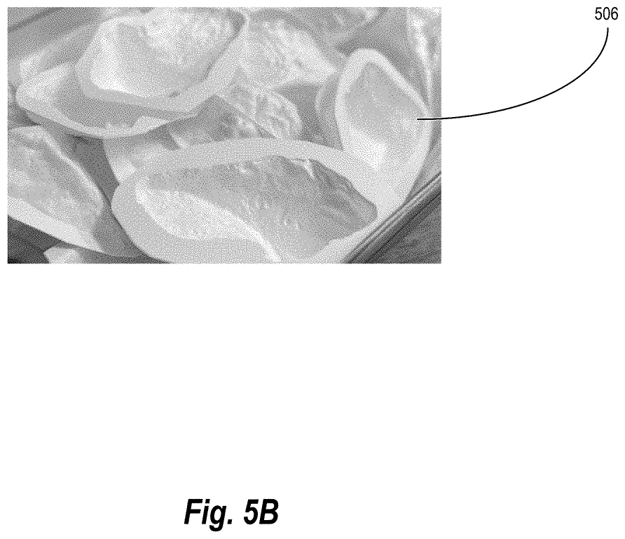

As mentioned, the disclosed method may utilize molds with various forms. FIGS. 5A-5B illustrate example molds in accordance with one or more embodiments. In particular, FIG. 5A illustrates an example mold 502 having surface texture 504 consistent with a chicken breast. For example, the surface texture can include contours and details corresponding to slaughtered meat. As illustrated, the mold 502 includes contours that correspond to meat fiber segments in slaughtered meat. Additionally, the mold 502 includes ridges that correspond to vascularization found in slaughtered meat.

Furthermore, and as illustrated in FIG. 5A, the mold 502 is made of a rigid polymer. The mold 502 may belong to a sheet of molds. For example, as depicted in FIG. 5A, the mold 502 is part of a sheet containing a total of six molds. A sheet of mold may include any number of individual molds. Furthermore, cell-mass mixtures may be sealed within molds that are part of a sheet or molds that have been cut out of a sheet.

FIG. 5B illustrates an example mold 506 of a smaller chicken nuggets. As illustrated in FIG. 5B, the mold 506 also includes surface texture and shape that mimic the texture and shape of nuggets made from slaughtered meat.

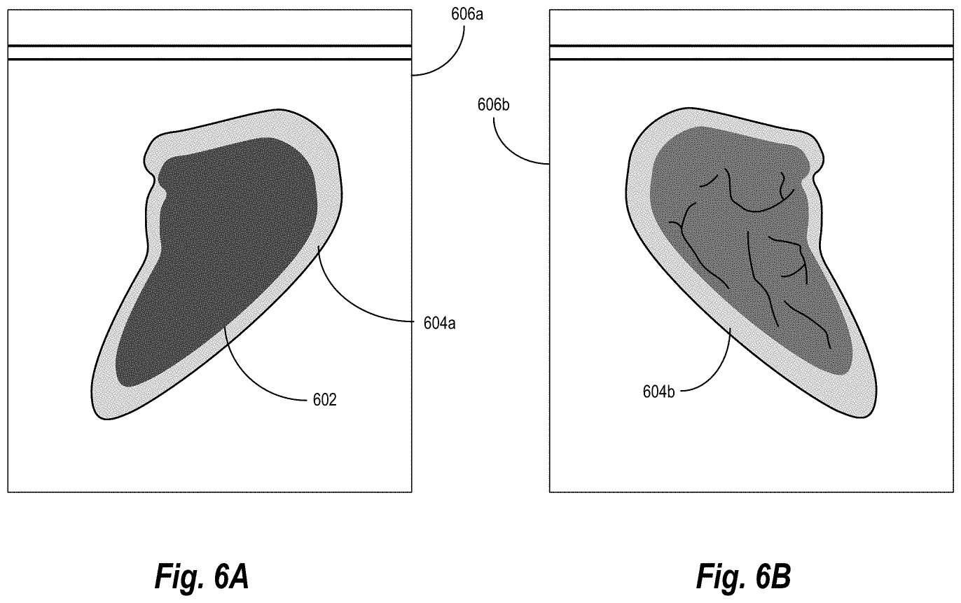

As noted above, in some embodiments, the disclosed method comprises covering the cell-mass mixture and the mold by enveloping the cell-mass mixture and the mold with a film. FIGS. 6A-6B illustrate an example film envelope that is vacuum sealed around a mold and cell-mass mixture. In particular, FIG. 6A illustrates a front view of a film envelope 606a and FIG. 6B illustrates a back view of a film envelope 606b.

As mentioned, FIG. 6A illustrates the front view of the film envelope 606a. A cell-mass mixture 602 is visible from the front view. In some embodiments, a mold 604a is also visible from the front view. The disclosed method utilizes the film envelope 606a by placing the mold 604a containing the cell-mass mixture 602 on a bottom film. A top film is placed over the mold 604a, the cell-mass mixture 602, and the bottom film. Air is removed from the film envelope 606a, and the film envelope 606a is sealed. For example, in some embodiments, the film envelope 606a is created by sealing three edges of the top film and the bottom film together. The fourth edge is sealed after air has been removed from the film envelope 606a.

FIG. 6B illustrates the back view of the film envelope 606b. As illustrated, the mold 604b, but not the cell-mass mixture, is visible from the back view. As mentioned, in some embodiments, the film envelope 606b and the mold 604b comprise heat and food-safe materials that may be heated as part of cooking the cell-mass mixture.

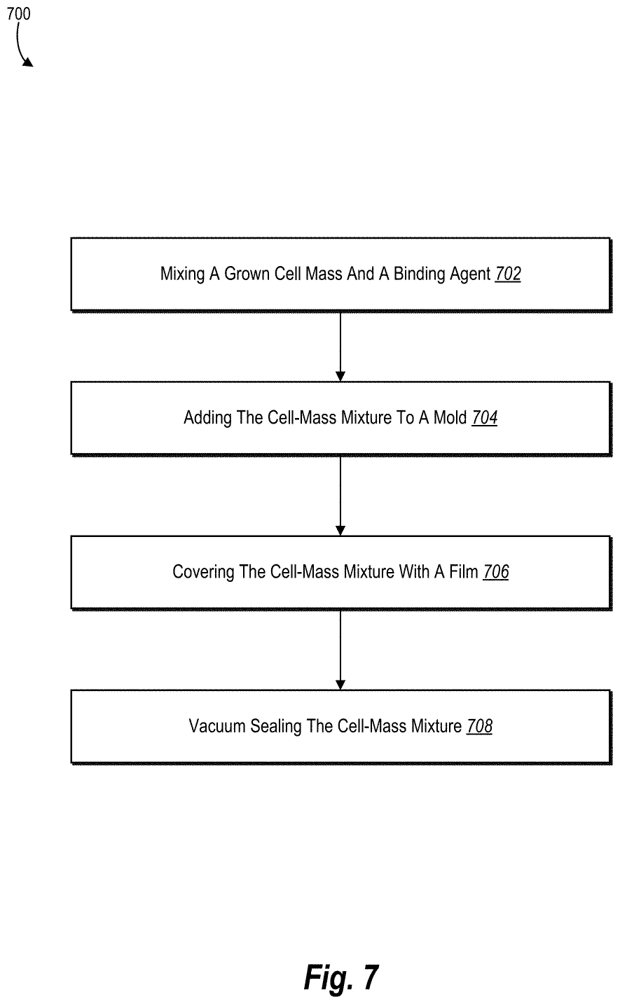

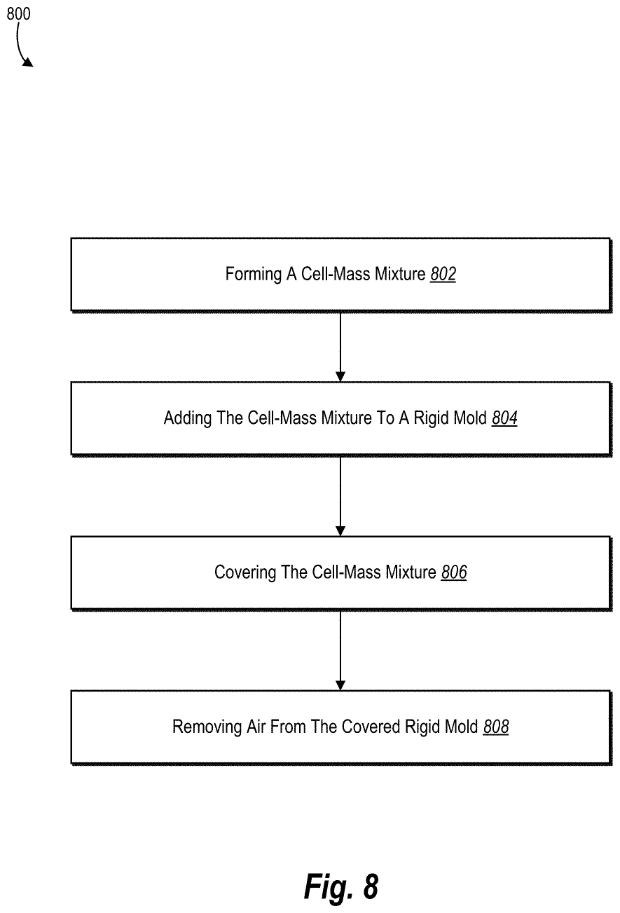

FIGS. 1-6B, the corresponding text, and the examples provide several different systems, methods, techniques, components, and/or devices relating to shaping a cell-meat mixture using vacuum sealing in accordance with one or more embodiments. In addition to the above description, one or more embodiments can also be described in terms of flowcharts including acts for accomplishing a particular result. FIGS. 7-8 illustrate such flowcharts of acts. The acts described herein may be repeated, rearranged, or performed in parallel with one another or in parallel with different instances of the same or similar acts.

FIG. 7 illustrates a flowchart of a series of acts 700. By way of overview, the series of acts 700 includes an act 702 of mixing a grown cell mass and a binding agent, an act 704 of adding the cell-mass mixture to a mold, an act 706 of covering the cell-mass mixture with a film, and an act 708 of vacuum sealing the cell-mass mixture.

The series of acts 700 includes the act 702 of mixing a grown cell mass and a binding agent. In particular, the act 702 comprises mixing a grown cell mass and a binding agent to form a cell-mass mixture. In some embodiments, the act 702 further comprises mixing the grown cell mass and the binding agent by combining the grown cell mass with transglutaminase. In some embodiments, the grown cell mass comprises raw cell-based meat. In some embodiments, the act 702 or the series of acts 700 further comprises mixing the grown cell mass and the binding agent in a vacuum blender.

The series of acts 700 includes the act 704 of adding the cell-mass mixture to a mold. In some embodiments, the act 704 further comprises adding the cell-mass mixture to a rigid mold to which the cell-mass mixture conforms. Furthermore, in some embodiments, adding the cell-mass mixture to the mold comprises adding the cell-mass mixture to a mold having surface texture to which an outer portion of the cell-mass mixture conforms. Similarly, in some cases, adding the cell-mass mixture to a rigid mold comprises adding the cell-mass mixture to a mold having surface texture to which an outer portion of the cell-mass mixture conforms. In some embodiments, adding the cell-mass mixture to the mold comprises: dividing the cell-mass mixture into portions based on weight; and depositing a portion of the portions into the mold.

The series of acts 700 includes the act 706 of covering the cell-mass mixture with a film. In particular, the act 706 comprises covering the cell-mass mixture and the mold with a film. In some embodiments, covering the cell-mass mixture and the mold with the film comprises: heating the film; and stretching the film over the mold.

As further illustrated in FIG. 7, the series of acts 700 includes the act 708 of vacuum sealing the cell-mass mixture. In particular, the act 708 comprises vacuum sealing the cell-mass mixture within the mold to shape the cell-mass mixture.

In some embodiments, the series of acts 700 further comprises an additional act of preparing the grown cell mass by dehydrating cells grown in suspension.

Furthermore, in some embodiments, the series of acts 700 includes an additional act of placing the vacuum sealed cell-mass mixture in a temperature-controlled environment for a threshold time.

FIG. 8 illustrates a series of acts 800. By way of overview, the series of acts 800 includes an act 802 of forming a cell-mass mixture, an act 804 of adding the cell-mass mixture to a rigid mold, an act 806 of covering the cell-mass mixture, and an act 808 of removing air from the covered rigid mold.

FIG. 8 illustrates the act 802 of forming a cell-mass mixture. In particular, the act 802 comprises mixing a raw grown cell mass and a binding agent to form a cell-mass mixture.

The series of acts 800 further includes the act 804 of adding the cell-mass mixture to a rigid mold. In particular, the act 804 comprises adding the cell-mass mixture to a rigid mold to which the cell-mass mixture conforms. Similarly, in some cases, adding the cell-mass mixture to a mold comprises adding the cell-mass mixture to a rigid mold to which the cell-mass mixture conforms. Further, in some embodiments, adding the cell-mass mixture to the rigid mold comprises: dividing the cell-mass mixture into portions based on weight; and depositing a portion of the portions into the rigid mold.

The series of acts 800 further includes the act 806 of covering the cell-mass mixture. In particular, the act 806 comprises covering the cell-mass mixture and the rigid mold with a film. In some embodiments, covering the cell-mass mixture and the rigid mold comprises: heating the film; and stretching the film over the rigid mold.

As further illustrated in FIG. 8, the series of acts 800 includes the act 808 of removing air from the covered rigid mold. In particular, the act 808 comprises removing air from the covered rigid mold comprising the cell-mass mixture.

In addition or in the alternative to the series of acts 800, in some embodiments a method includes mixing the raw grown cell mass and the binding agent by combining the raw grown cell mass with transglutaminase. Further, in some embodiments, the series of acts 800 further comprises an additional act of preparing the raw grown cell mass by dehydrating cells grown in suspension.

In addition or in the alternative to the series of acts 700 in FIG. 7 or the series of acts 800 in FIG. 8, this disclosure includes an apparatus for shaping raw cell-based meat. In some cases, the apparatus includes a portioner for depositing a cell-mass mixture; a rigid mold for holding the cell-mass mixture; a film applicator for covering the rigid mold and the cell-mass mixture; and a vacuum sealer for sealing and shaping the cell-mass mixture to the rigid mold.

In certain embodiments, the portioner: divides the cell-mass mixture into portions; and deposits the portions. Further, in some cases, the rigid mold is made of a heat safe material in which the cell-mass mixture may be cooked. As suggested above, in certain embodiments, the rigid mold has surface texture to which an outer portion of the cell-mass mixture conforms.

In accordance with common practice, the various features illustrated in the drawings may not be drawn to scale. The illustrations presented in the present disclosure are not meant to be actual views of any particular apparatus (e.g., device, system, etc.) or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absent a showing that the terms “first,” “second,” “third,” etc., connote a specific order, these terms should not be understood to connote a specific order. Furthermore, absent a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

The present invention may be embodied in other specific forms without departing from its spirit or essential characteristics. Indeed, the described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

1. A method for shaping a cell-based product comprising:
moisture adjusting a raw grown cell mass to increase a protein content of the raw grown cell mass;
forming a cell-mass mixture comprising the moisture adjusted raw grown cell mass and a binding agent;
adding the cell-mass mixture to a mold having a surface texture resembling a surface of a non-human animal body part cut in whole from raw slaughtered meat;
covering the cell-mass mixture and the mold with a film;
vacuum sealing the cell-mass mixture within the mold to form a vacuum sealed cell-mass mixture that conforms to the surface texture resembling the surface of the non-human animal body part cut in whole from the raw slaughtered meat; and
setting the vacuum sealed cell-mass mixture for a threshold time to cause cells within the vacuum sealed cell-mass mixture to bind together and retain a shape from the mold resembling the surface of the non-human animal body part.

2. The method of claim 1, further comprising:
moisture adjusting the raw grown cell mass until a protein content of the raw grown cell mass is sufficient for a binding agent to bind proteins of the raw grown cell mass.

3. The method of claim 1, wherein the mold comprises a rigid mold to which the shape of the cell-mass mixture conforms during vacuum sealing.

4. The method of claim 1, wherein the surface texture further comprises striations corresponding to meat fiber segments in the raw slaughtered meat.

5. The method of claim 1, further comprising:
removing a shaped cell-mass mixture from the mold after the vacuum sealed cell-mass mixture has set for the threshold time; and
cooking the shaped cell-mass mixture.

6. The method of claim 2, wherein the protein content of the moisture adjusted raw grown cell mass is sufficient for the binding agent to bind proteins without adding protein additives.

7. The method of claim 1, wherein the surface texture further comprises contours that correspond to meat fiber segments in the raw slaughtered meat.

8. The method of claim 1, wherein the mold resembles a boneless non-human animal body part cut in whole from the raw slaughtered meat.

9. The method of claim 1, wherein setting the vacuum sealed cell-mass mixture for the threshold time comprises placing the vacuum sealed cell-mass mixture in a temperature-controlled environment.

10. The method of claim 1, wherein the threshold time comprises a time required for the cell-mass mixture to retain the shape from the mold.

11. A method for shaping a comestible cell-based product comprising:
moisture adjusting a raw grown cell mass to increase a protein content of the raw grown cell mass;
forming a cell-mass mixture comprising the moisture adjusted raw grown cell mass and a binding agent;
adding the cell-mass mixture to a rigid mold comprising a rigid polymer to which the raw grown cell mass of the cell-mass mixture conforms;
covering the cell-mass mixture and the rigid mold with a flexible film; and
removing air from the covered rigid mold comprising the cell-mass mixture to conform the raw grown cell mass of the cell-mass mixture to the rigid mold.

12. The method of claim 11, further comprising moisture adjusting the raw grown cell mass until a protein content of the raw grown cell mass is sufficient for a binding agent to bind proteins of the raw grown cell mass.

13. The method of claim 11, wherein the flexible film comprises a flexible polymer.

14. The method of claim 11, wherein covering the cell-mass mixture and the rigid mold comprises:
heating the flexible film; and
stretching the flexible film over the rigid mold.

15. The method of claim 11, further comprising:
moisture adjusting the raw grown cell mass by dehydrating the raw grown cell mass until a protein content of the raw grown cell mass satisfies a threshold protein content of 15%; and
forming the cell-mass mixture by combining the dehydrated raw grown cell mass with transglutaminase as the binding agent.

16. The method of claim 11, further comprising setting the covered cell-mass mixture for a threshold time to cause cells within the covered cell-mass mixture to bind together and retain a shape of the rigid mold.

17.-20. (canceled)

21. The method of claim 11, further comprising heating the rigid mold and the covered cell-mass mixture to cook the cell-mass mixture within the rigid mold.

22. The method of claim 11, wherein removing the air from the covered rigid mold comprising the cell-mass mixture comprises removing air bubbles from the cell-mass mixture within the covered rigid mold.

23. The method of claim 11, wherein the raw grown cell mass comprises adherent cells grown while attached to a surface within a bioreactor.

24. The method of claim 11, wherein the raw grown cell mass comprises suspension cells grown in an agitated growth medium within a bioreactor.
